(19) United States
(12) Patent Application Publication
(10) Pub. No.: US 2011/0287464 A1
(43) Pub. Date: Nov. 24, 2011

(54) Chromogenic plating media for the identification of Enterobacter sakazakii

(76) Inventor: Lawrence Restaino, Elburn, IL (US)

(21) Appl. No.: 13/136,106

(22) Filed: Jul. 21, 2011

Related U.S. Application Data
(63) Continuation of application No. 11/128,741, filed on May 13, 2005, now abandoned.

(51) Int. Cl. C12Q 1/34

(52) U.S. Cl. 435/18

(57) ABSTRACT

A plating medium for identification of Enterobacter sakazakii bacteria having a carbohydrate, but Enterobacter sakazakii bacteria being incapable of fermenting any carbohydrate in the medium. The medium also contains a pH indicator dye that changes the color of the medium from a first color to a second color when the pH changes, first and second chromogenic substrates that react to alpha-glucosidase and beta cellobiosidase enzymes, respectively, to produce a third color in the medium, and agar to solidify the mixture. Microorganisms that ferment the carbohydrate but do not produce alpha-glucosidase or beta-cellobiosidase will produce colonies of the second color, microorganisms that produce alpha-glucosidase and/or beta-cellobiosidase including Enterobacter sakazakii bacteria will produce colonies of the third color, and microorganisms that ferment the carbohydrate and produce alpha glucosidase and/or beta-cellobiosidase will produce colonies of a fourth color which is the color that results from mixing the second and third colors.

This application is a continuation application of U.S. patent application No. 11/128,741 filed on May 13, 2005.

This invention relates to devices for identifying one particular microorganism from an environment containing a mixture of microorganisms. More specifically, the present invention relates to plating media for the rapid detection and identification of Enterobacter sakazakii bacteria from an environment containing a plurality of microorganisms.

### BACKGROUND OF THE INVENTION

Enterobacter sakazakii was described as a bacterial species in 1980. It was formerly known as yellow pigmented Enterobacter cloacae. As reported by Leuschner, Baird, Donald and Cox in “A Medium for the Presumptive Detection of Enterobacter sakazakii in Infant Formula,” Food Microbiology 21 (2004), 527-533, Enterobacter sakazakii has been implicated in a severe form of neonatal meningitis with a high mortality rate. It is reported that many newborns with Enterobacter sakazakii meningitis die within days of infection, and that the case-fatality rates vary between 40 and 80%, Nazarowec-White and Farber, “Enterobacter sakazakii: A Review, International Journal of Food Microbiology 34 (1997) 103-113. While a reservoir for Enterobacter sakazakii bacteria is unknown, reports have suggested that powdered milk-based infant formula may be a vehicle for infection. There have also been reported cases of infection in adults caused by Enterobacter sakazakii bacteria.

Accordingly, there is a clear need for a rapid and accurate device for detecting and identifying Enterobacter sakazakii bacteria in food and on surfaces. Researchers have used nutrient agar plating media that is responsive to the alpha-glucosidase enzyme prior to the present invention, but such media are subject to the production of false negatives, have been time consuming, and produce plates that are difficult to read and analyze because of colonies of unwanted microorganisms. Accordingly, such media have serious drawbacks for isolating and enumerating Enterobacter sakazakii from foods or the diagnosis of infections in newborns and adults. The article by Leuschner, Baird, Donald and Cox, supra, describes the detection and identification of Enterobacter sakazakii in infant formula using a nutrient agar supplemented with the enzyme substrate 4-methyl-umbelliferyl-alpha-D-glucoside. This plating medium will produce a substantial number of false negatives, because some Enterobacter sakazakii isolates can not utilize the substrate 4-methyl-umbelliferyl-alpha-D-glucoside. Further, the detection and identification process was excessively time consuming, requiring separate enrichment and testing steps.

### SUMMARY OF INVENTION

It is an object of the present invention to provide a culture plating medium for the presumptive detection and identification of Enterobacter sakazakii which is not inherently subject to false negative results.

It is also an object of the present invention to provide a culture plating medium for the presumptive detection and identification of Enterobacter sakazakii bacteria which produces colonies and can be observed and enumerated in a shorter time period than plating of the prior art.

It is also an object of this invention to provide a plating medium for the presumptive detection and identification of Enterobacter sakazakii bacteria that produces colonies that can be easily read and differentiated from other non Enterobacter sakazakii colonies.

The inventor achieves the objects of the present invention by providing a culture medium that displays a first color and is provided with ample nutrients to promote the growth of Enterobacter sakazakii bacteria. This medium is also provided with a first substrate that responds to the alpha-glucosidase enzyme to color the medium with a second color. Further, the medium has at least one carbohydrate and an indicator dye that respond to a change in the pH of the medium to release a dye into the medium of a third color. The carbohydrate is selected from a class of carbohydrates that are not fermented by Enterobacter sakazakii, thereby assuring that Enterobacter sakazakii bacteria will produce colonies in the medium of the second color. Microorganisms that ferment the carbohydrate produce unwanted colonies, and these colonies appear as the third color. In order to respond to the enzymes produced by all of the Enterobacter sakazakii bacteria, the medium is provided with a second substrate that responds to beta-cellobiosidase produced by Enterobacter sakazakii to produce the same second color in the media.

The media differentiates between four different groups of microorganisms. First, those microorganisms that do not ferment any of the carbohydrates and do not use the chromogenic substrates produce colonies in the medium of the first color (the color of the medium). Second, those microorganisms that ferment a carbohydrate, but do not use the chromogenic substrates, produce colonies in the media of the second color. Third, those microorganisms that use a chromogenic substrate, but do not ferment any of the carbohydrates form colonies in the medium of a third color (Enterobacter sakazakii are in this group). Fourth, those microorganisms that ferment a carbohydrate and use a chromogenic substrate produce colonies in the medium of a fourth color which is the color resulting from blending together the second and third colors. By selecting the first, second and third colors to be contrasting colors, all four colors may be contrasting, thus facilitating reading and enumerating of the colonies on the surface of the processed and incubated plate.

The present invention also inhibits unwanted microorganisms from growing on the medium. Inhibitors for gram positive microorganisms, Proteus and Pseudomonas are ingredients of the medium. To be an effective inhibitor, an inhibitor must not inhibit the microorganism of interest, and prior to the present invention no effective inhibitor for use in agar media for the detection and identification of Enterobacter sakazakii was known for Proteus. The inventor discovered that vancomycin and cefsulodin function as inhibitors of Proteus and Pseudomonas, respectively, and do not adversely effect the growth of Enterobacter sakazakii in a nutrient medium.

### DETAILED DESCRIPTION OF THE INVENTION

The plating medium of the present invention contains nutrients to promote the growth of Enterobacter sakazakii, especially protein. In the preferred embodiment, a mixture of tryptone, peptone G, proteose-peptone and yeast extract is used, but it is to be understood that each of these ingredients can be separately used, used in other combinations, or other nutrients can be used.

The inventor's preferred identification system for Enterobacter sakazakii utilizes a solid plating medium containing a substrate that reacts to the alpha-glucosidase enzyme. The preferred substrate is 5-Bromo-4-Chloro-3-Indoxyl-alpha-D-Glucopyranoside which produces a dark blue precipitate when cleaved. Other substrates suitable for practicing the present invention are 4-Methylumbelliferyl-alpha-D-Glucopyranoside, 2-Naphthyl-alpha-D-Glucopyranoside, 4-Nitrophenyl-alpha-D-Glucopyranoside, 5-Bromo-6-Chloro-3-Indoxyl-alpha-D-Glucopyranoside, 6-Chloro-3-Indoxyl-alpha-D-Glucopyranoside, 3-Indoxyl-alpha-D-Glucopyranoside, and 2-Nitrophenyl-alpha-D-Glucopyranoside.

While Enterobacter sakazakii bacteria produce alpha-glucosidase, not all Enterobacter sakazakii are detected by a plating medium with only an alpha-glucosidase substrate, thus resulting in false negatives. To overcome this deficiency, the media of the present invention incorporate a second substrate that responds to the beta cellobiosidase enzyme. Almost 100 percent of the Enterobacter sakazakii produce cellobiosidase. In the preferred embodiment of this invention, the medium contains a second substrate which is cleaved by the cellobiosidase enzyme; the second substrate producing the same third color as the first substrate, thus eliminating false negative responses to Enterobacter sakazakii bacteria. The second substrate in the preferred embodiment is 5-Bromo-4-Chloro-3-Indoxyl-beta-D-Cellobioside. Other substrates that respond to the beta-cellobiosidase enzymes are 4-Methylumbelliferyl-beta-D Cellobioside, 2-Napthyl-beta-D-Cellobioside, 4-Nitrophenyl-beta-D-Cellobiosidase, 2-Nitrophenyl-beta-D-Cellobiosidase, 5-Bromo-6-Chloro-3-Indoxyl-beta-D-Cellobioside, 6-Chloro-3-Indoxyl-beta-D-Cellobioside, and 3-Indoxyl-beta-D-Cellobioside.

The preferred detection system using 5-Bromo-4-Chloro-3-Indoxyl-alpha-D-Glucopyranoside and 5-Bromo-4-Chloro-3-Indoxyl-beta-D-Cellobioside will respond to alpha-glucosidase and beta-cellobiosidase enzymes which will eliminate false negatives.

The differentiation system employs one or more carbohydrates that are not metabolized by Enterobacter sakazakii bacteria and are selected from the group sorbitol, adonitol, and D-arabitol. In the preferred embodiment, all three carbohydrates are utilized. The differentiation system also uses an indicator dye which responds by releasing a dye into the plating medium to change the color of the medium responsive to a change in the pH of the medium, the changed color being significantly different from the color of the medium and the color produced on activation by the substrate or substrates. In the preferred embodiment, the indicator dye is phenol red which produces a yellow color responsive to an acid change in the pH of the medium. In the preferred embodiment, the pH of the medium is adjusted to 6.8 to 7.0. Sodium chloride is also added to the medium for osmolarity purposes.

Also in the preferred embodiment of the present invention, inhibitors that will not inhibit the growth of Enterobacter sakazakii are employed. An inhibitor for gram positive bacteria is utilized, and in the preferred embodiment it is bile salts #3. Other inhibitors of gram positive bacteria can also be employed.

The medium of the preferred embodiment preferably contains a growth inhibitor for Proteus sp, but known inhibitors of Proteus sp also inhibit the growth of Enterobacter sakazakii. The inventor has found that vancomycin will retard Proteus sp. without retarding the growth of Enterobacter sakazakii, and in the preferred embodiment of the medium of the present invention vancomycin hydrochloride is incorporated for this purpose. Also, the medium contains sodium cefsulodin hydrate to inhibit Pseudomonas and Aeromonas bacteria without affecting the growth of Enterobacter sakazakii.

The preferred embodiment of the plating medium contains the ingredients in the proportions set forth in the following Table I.

TABLE I

| MATERIAL | MEASUREMENT | |
|---|---|---|
| Tryptone | 4.00 | grams/liter |
| Peptone G | 4.35 | grams/liter |
| Proteose-peptone | 3.0 | grams/liter |
| Yeast extract | 6.0 | grams/liter |
| Sodium Chloride | 5.0 | grams/liter |
| D-Arabitol | 5.00 | grams/liter |
| Adonitol | 8.00 | grams/liter |
| Sorbitol | 10.0 | grams/liter |
| Phenol red | 0.10 | grams/liter |
| Bile salts #3 | 1.25 | grams/liter |
| 5 bromo-4 chloro-3 indoxyl-alpha-D-glucopyranoside | 0.15 | grams/liter |
| 5-bromo-4-chloro-3-indoxyl-β-D-cellobioside | 0.15 | grams/liter |
| Agar | 15.00 | grams/liter |
| Sodium cefsulodin hydrate | 0.006 | grams/liter |
| Vancomycin hydrochloride | 0.008 | grams/liter |

Except for sodium cefsulodin hydrate and vancomycin hydrochloride, the ingredients are mixed in any order, the pH adjusted to 6.9 to 7.0, boiled to sterilize the mixture, and the mixture is permitted to cool to room temperature. Thereafter, sterile sodium cefsulodin hydrate and vancomycin hydrochloride at room temperature are added aseptically to the other ingredients. The composition is then poured into plates and permitted to dry for 48 to 72 hours in the dark, and the plates are then ready to be used. Storage time of poured plates is as much as 60 days at 2 to 8 degrees Celsius.

The process of the present invention requires a plate or mass of the plating medium to be inoculated with the test sample, and the inoculated mass is then incubated for a period of time to permit growth of the microorganisms in the test sample to observable colonies. The inventor has found that with the preferred plating medium described above, a period of 24 hours of incubation is sufficient time for Enterobacter sakazakii colonies present in a test sample to grow into colonies that are readily observable with the naked eye. It is believed that the abundant growth of microorganisms in the preferred plating medium is due to the nutrients provided by the tryptone, peptone-G, proteose-peptone, yeast extract, sorbitol, adonitol and D-arabitol. The surface of the plating medium mass is then assayed and the presence and number of blue-black to blue-grey with black precipitate colonies recorded. Also, the presence of clear to white or yellow to green colored colonies is noted as an indication of microorganisms other than Enterobacter sakazakii.

It is to be noted that no special equipment is required to observe the incubated mass of plating medium. The time required to note the number and presence of blue-black to blue-grey with black precipitate colonies is far less than required when other colonies are present. Also, there are no ingredients in the plating medium that are especially costly. Hence, an assay of a test sample may be made at reduced cost from assays made with prior plating media.

The following Table II sets forth examples of use of the plating medium described in Table I by the process described above, the test sample containing the microorganism shown in the left column and the observed colonial description being set forth in the right column.

TABLE II

| Organism | Colonial Morphology |
|---|---|
| Enterobacter sakazaki | Blue-black raised to domed colonies 1-2.0 mm diameter ± clear |
| | rings; One strain <1.0 mm diameter and one strain with blue-gray |
| | colonies |
| Enterobacter aerogenes | Yellow domed colonies 1-1.5 mm diameter with clear ring |
| Enterobacter gergoviae | White to light gray domed colonies 1-2.0 mm diameter with clear |
| | rings |
| Pantoea species (2) strains | White to yellow domed colonies 1-1.5 mm diameter with clear ring |
| Escherichia coli | White to yellow domed colonies 2.0 mm diameter with clear rings |
| Escherichia coli | White raised colonies 2.0 mm diameter with clear rings |
| Escherichia coil sorbitol positive | White to yellow domed colonies 2.0 mm diameter with clear rings |
| Escherichiia coli H₂S positive | White or yellow to gray domed colonies 2.0 mm diameter with clear |
| | rings |
| Escherichia coli O157: H7 (12 strains) | Clear to white and either flat or raised colonies 1-2.0 mm diameter ± |
| | clear rings |
| Escherichia hermanii | Clear to yellow raised or domed colonies 1-1.5 mm diameter ± clear |
| | rings |
| Citrobacter freundii | Clear to white domed colonies 2.0 mm diameter with clear rings |
| Klebsiella ozanae | Green to yellow domed colonies 1-1.5 mm diameter with clear rings |
| Klebsiella pneumoniae | Yellow to green domed colonies 1.0 mm diameter without clear |
| | rings |
| Morganella morganii | Clear flat colonies 1.0 mm diameter without clear rings |
| Morganella reltgeri | Yellow raised colonies 2.0 mm diameter with clear rings |
| Providencia stuartii | Clear flat colonies 1-1.5 mm diameter without clear rings |
| Salmonella (5 species) | White to yellow domed colonies 1-2 mm diameter with clear rings |
| Shigella dysenteria | Clear to white domed colonies 1-1.5 mm diameter with clear rings |
| Shigella flexneri | Clear to white raised colonies 1-1.5 mm diameter with clear rings |
| Shigella sonnet (3 strains) | Blue-black or blue-gray flat or raised colonies 1.5 · 2.0 mm diameter |
| | with clear rings |
| Shigella boydii | Clear to white raised colonies 1.0 mm diameter with no clear rings |
| Pseudomonas aeruginosa | Clear flat colonies <1.0 mm diameter with no clear rings |
| Hafnia alvei | Clear flat colonies 1.0 mm diameter with no clear rings |
| Listeria monocytogenes | No growth for all tested strains |
| Listeria grayii | |
| Listeria ivanovii | |
| Listeria innocua | |
| Bacillus cereus | |
| Streptococcus avium | |
| Enterococcus fitecalis | |
| Enterococcus faecium | |
| Staphylococcus aureus | |

Those skilled in the art will devise other methods of utilizing the plating media of the present invention, and other plating media than those specifically described in the foregoing specification within the scope of the present invention. It is therefore intended that the scope of the present invention be not limited by the foregoing specification, but rather only by the appended claims.

1. An isolation plating medium for the presumptive identification of Enterobacter sakazakii bacteria from a sample that also contains other microorganisms, said medium displaying-a first color, comprising a mixture of at least one nutrient ingredient for promoting growth of Enterobacter sakazakii bacteria, at least one carbohydrate for fermentation by bacteria other than Enterobacter sakazakii bacteria, all carbohydrates in said medium being selected from carbohydrates that are not fermented by Enterobacter sakazakii, a pH indicator dye that changes the color of the plating medium from the first color to a second color at the site of a fermentation reaction that changes the pH of the mediums, a chromogenic substrate for reacting with beta-cellobiosidase enzymes to produce precipitate of a third color at the sites of said reactions in the plating medium, and a sufficient mass of an agent to solidify the mixture, whereby microorganisms that are in the medium and do not ferment the carbohydrate and do not react with the substrate produce colonies in the medium of the first color, microorganisms that are in the medium and ferment the carbohydrate but do not react with the substrate produce colonies in the plating medium of the second color, Enterobacter sakazakii and other microorganisms that are in the medium and react with the substrate but do not react with the carbohydrate-produce colonies in the plating medium of the third color, and microorganisms that are in the medium and ferment the carbohydrate and react with the substrate produce colonies in the plating medium of a fourth color which is the color that results from mixing of the second and third colors, the first, second, third and fourth colors contrasting with each other.

2. The isolation plating medium for the presumptive identification of Enterobacter sakazakii bacteria as in claim 1 wherein the mixture includes a chromogenic substrate for reacting to alpha glucosidase enzymes to produce at the site of the alpha-glucosidase enzyme and substrate reaction the same third color in the plating medium as the beta-cellobiosidase enzyme and substrate reaction.

3. The isolation plating medium for the presumptive identification of Enterobacter sakazakii bacteria as in claim 2 wherein the chromogenic substrate for reacting to alpha glucosidase enzymes is selected from the group consisting of 5-Bromo-4-Chloro-3-Indoxyl-alpha-D-Glucopyranoside, 4-Methylumbelliferyl-alpha-D-Glucopyranoside, 2-Naphthyl-alpha-D-Glucopyranoside, 4-Nitrophenyl-alpha-D-Glucopyranoside, 5-Bromo-6-Chloro-3-Indoxyl- alpha-D-Glucopyranoside, 6-Chloro-3-Indoxyl-alpha-D-Glucopyranoside, 3-Indoxyl-alpha-D-Glucopyranoside, and 2-Nitrophenyl-alpha-D-Glucopyranoside.

4. The isolation plating medium for the presumptive identification of Enterobacter sakazakii bacteria as in claim 1 wherein the chromogenic substrate for reacting with the beta-cellobiosidase enzymes is selected from the group consisting of 5-Bromo-4-Chloro-3-Indoxyl-beta-D-Cellobioside, 4-Methlumbelliferryl-beta-D Cellobioside, 2-Napthyl-beta-D-Cellobioside, 4-Nitrophenyl-beta-D-Cellobiosidase, 2-Nitrophenyl-beta-D-Cellobiosidase, 5-Bromo-6-Chloro-3-Indoxyl-beta-D-Cellobioside, 6-Chloro-3-Indoxyl-beta-D-Cellobioside, and 3-Indoxyl-beta-D-Cellobioside.

5. The isolation plating medium for the presumptive identification of Enterobacter sakazakii bacteria as in claim 1 wherein the carbohydrate is selected from the group consisting of sorbitol, adonitol, and D-arabitol.

6. The isolation plating medium for the presumptive identification of Enterobacter sakazakii bacteria as in claim 5-wherein the indicator dye is phenol red.

7. (canceled)

8. The isolation plating medium for the presumptive identification of Enterobacter sakazakii bacteria as in claim 1 wherein the one or more nutrient ingredient is selected from the group consisting of tryptone, peptone G, proteose-peptone and yeast extract.

9. The isolation plating medium for the presumptive identification of Enterobacter sakazakii bacteria as in claim 1 wherein the medium includes one or more ingredients to retard the growth of one or more bacteria selected from the group consisting of gram positive microorganisms, Proteus sp., Pseudomonas sp. and Aeromonas sp.

10. The isolation plating medium for the presumptive identification of Enterobacter sakazakii bacteria as in claim 9 wherein the ingredient to retard growth of Proteus sp. is vancomycin.

11. The isolation plating medium for the presumptive identification of Enterobacter sakazakii bacteria as in claim 9 wherein the ingredient for retarding growth of Pseudomonas sp. and Aeromonas sp. is cefsulodin.

12. (canceled)

13. An isolation plating medium for the presumptive identification of Enterobacter sakazakii bacteria from a sample that also contains other microorganisms, said medium displaying a first color, comprising a mixture of at least one nutrient ingredient for promoting growth of Enterobacter sakazakii bacteria, at least one carbohydrate for fermentation by bacteria other than Enterobacter sakazakii bacteria, all carbohydrates in said medium being selected from carbohydrates that are not fermented by Enterobacter sakazakii, a pH indicator dye that changes the color of the plating medium from the first color to a second color at the site of a fermentation reaction that changes the pH of the medium, a chromogenic substrate for reacting with beta-cellobiosidase enzymes, said substrate being selected from the group consisting of 5-Bromo-4-Chloro-3-Indoxyl-beta-D-Cellobioside, 4-Methlumbelliferryl-beta-D Cellobioside, 2-Napthyl-beta-D-Cellobioside, 4-Nitrophenyl-beta-D-Cellobiosidase, 2-Nitrophenyl-beta-D-Cellobiosidase, 5-Bromo-6-Chloro-3-Indoxyl-beta-D-Cellobioside, 6-Chloro-3-Indoxyl-beta-D-Cellobioside, and 3-Indoxyl-beta-D-Cellobioside, a chromogenic substrate for reacting to alpha glucosidase enzymes, said substrate being selected from the group consisting of 5-Bromo-4-Chloro-3-Indoxyl-alpha-D-Glucopyranoside, 4-Methylumbelliferyl-alpha-D-Glucopyranoside, 2-Naphthyl-alpha-D-Glucopyranoside, 4-Nitrophenyl-alpha-D-Glucopyranoside, 5-Bromo-6-Chloro-3-Indoxyl-alpha-D-Glucopyranoside, 6-Chloro-3-Indoxyl-alpha-D-Glucopyranoside, 3-Indoxyl-alpha-D-Glucopyranoside, and 2-Nitrophenyl-alpha-D-Glucopyranoside, the chromogenic substrate for reacting with beta-cellobiosidase enzymes and the chromogenic substrate for reacting with alpha glucosidase enzymes releasing precipitates of the same third color in the plating medium in response to enzyme reactions, and a sufficient mass of agar to solidify the mixture, whereby microorganisms in the medium that do not ferment the carbohydrates and do not react with a chromogenic substrate produce colonies of the first color, microorganisms in the medium that ferment the carbohydrate but do not produce alpha-glucosidase or beta cellobiosidase enzymes produce colonies in the plating medium of the second color, Enterobacter sakazakii and other microorganisms in the plating medium that react with a substrate and do not ferment a carbohydrate produce colonies in the plating medium of the third color, and microorganisms that ferment the carbohydrate and react with a substrate produce colonies in the plating medium of a fourth color which is the color that results from mixing the second and third colors, the first, second, third and fourth colors contrasting with each other.

14. The isolation plating medium for the presumptive identification of Enterobacter sakazakii bacteria as in claim 13 wherein the at least one carbohydrate is selected from the group consisting of sorbitol, adonitol, and D-arabitol.

15. The isolation plating medium for the presumptive identification of Enterobacter sakazakii bacteria as in claim 14 wherein the chromogenic substrate for reacting with beta-cellobiosidase enzymes is 5-Bromo-4-Chloro-3-Indoxyl-beta-D-Cellobioside, and the chromogenic substrate for reacting to alpha glucosidase enzymes is 5-Bromo-4-Chloro-3-Indoxyl-alpha-D-Glucopyranoside

16. (canceled)

17. (canceled)

18. The isolation plating medium for the presumptive identification of Enterobacter sakazakii bacteria as in claim 13 wherein the medium includes vancomycin to retard the growth of Proteus sp.

19. The isolation plating medium for the presumptive identification of Enterobacter sakazakii bacteria as in claim 13 wherein the medium includes cefsulodin to retard the growth of Pseudomonas sp. and Aeromonas sp.

20-21. (canceled)

22. An isolation plating medium for the presumptive identification of Enterobacter sakazakii bacteria from a sample that also contains other microorganisms, said medium displaying a first color, comprising a mixture of at least one nutrient ingredient for promoting growth of Enterobacter sakazakii bacteria, at least one carbohydrate selected from the group consisting of sorbitol, adonitol, and D-arabitol, all carbohydrates in said medium being selected from carbohydrates that are a not fermented by Enterobacter sakazakii, a pH indicator dye that changes the color of the plating medium from the first color to a second color at the site of a fermentation reaction that changes the pH of the medium, a chromogenic substrate for reacting with beta-cellobiosidase enzymes and a chromogenic substrate for reacting with alpha-glucosidase enzymes, each of said chromogenic substrates releasing percipitate of the same third color into the medium at the site of a reaction between an enzyme and a substrate, and a sufficient mass of agar to solidify the mixture, whereby microorganisms that are in the medium and do not ferment the carbohydrate and do not react with the substrate produce colonies in the medium of the first color, microorganisms that are in the medium and ferment the carbohydrate but do not react with a substrate produce colonies in the plating medium of the second color, Enterobacter sakazakii and other microorganisms that are in the media and react with the substrate but do not react with the carbohydrate produce colonies in the plating medium of the third color, and microorganisms that are in the medium and ferment a carbohydrate and react with a substrate produce colonies in the plating medium of a fourth color which is the color that results from mixing of the second and third colors, the first, second, third and fourth colors contrasting with each other.
